(19) United States
(12) Patent Application Publication
(10) Pub. No.: US 2024/0041787 A1
(43) Pub. Date: Feb. 8, 2024

(54) PHOTOSENSITIZER MOLECULE AND USE THEREOF IN INCREASING TUMOR RETENTION TIME AND ENHANCING TREATMENT OF LARGE-VOLUME TUMORS

(71) Applicant: DALIAN UNIVERSITY OF TECHNOLOGY, Dalian, Liaoning (CN)

(72) Inventors: Wen SUN, Dalian, Liaoning (CN); Dongmei XI, Dalian, Liaoning (CN); Xiaojun PENG, Dalian, Liaoning (CN); Jiangli FAN, Dalian, Liaoning (CN)

(21) Appl. No.: 18/256,298

(22) PCT Filed: Sep. 26, 2022
(86) PCT No.: PCT/CN2022/121296

§ 371 Date: Jun. 7, 2023

(30) Foreign Application Priority Data
Oct. 14, 2021 (CN) 202111199305.6

(51) Int. Cl. A61K 9/51; A61K 41/00; C07F 5/02; A61K 9/00; A61P 35/00

(52) U.S. Cl. CPC A61K 9/5146; A61K 41/0057; C07F 5/022; A61K 9/0019; A61P 35/00

(57) ABSTRACT

A photosensitizer molecule increases the retention time thereof in a tumor and the enhancement of the therapy for large-volume tumors. AN-BDP is synthesized by means of introducing anthracene into BODIPY-meso, and due to the strong intermolecular π-π interaction of anthracene, AN-BDP and an amphiphilic block copolymer DSPE-PEG₂₀₀₀ are self-assembled into stable nanoparticles AN-BDP NPs. By means of anthracene, AN-BDP can be excited to a triplet excited state under light irradiation and acts with O₂ to generate ¹O₂. AN-BDP NPs are enriched at the tumor site and retained for about 10 days after tail vein injection. In a mouse tumor model having a conventional volume tumor, AN-BDP NPs can completely inhibit the growth of the tumor by means of one-time tail vein injection and one-time photoirradiation therapy.

### TECHNICAL FIELD

The present invention relates to the field of nanomaterials, in particular to a photosensitizer molecule and a use thereof in increasing tumor retention time thereof and enhancing the treatment of large-volume tumors.

### BACKGROUND

Photodynamic therapy (PDT), due to its minimal invasiveness and high spatiotemporal precision, results in considerable attention. As a key part of PDT, photosensitizer transfers absorbed energy to surrounding oxygen and generates reactive oxygen species (ROS), and then further reacts with nearby biomacromolecules (such as lipid, protein, and DNA) to kill cancer cells and complete PDT. Therefore, effective enrichment of the photosensitizer at tumor sites is a prerequisite for PDT of cancer patients, and its tumor retention effect directly determines whether the treatment is accurate and effective. However, in the process of in vivo PDT, most organic small-molecule photosensitizers are rapidly cleared from the bloodstream usually within hours or even ten minutes, so that the photosensitizers cannot be effectively enriched and retained at the tumor sites, thereby resulting in low PDT efficiency. Therefore, it is urgent to improve the retention time of photosensitizers at tumor sites, which is of great significance to ensure the accurate and efficient PDT in vivo.

One of the current major strategies to prolong blood circulation and tumor retention time is to nanoengineering the photosensitizers, such as organic polymer nanoparticles (NPs), liposomes or inorganic nanomaterials. Researchers can well control and optimize tumor retention time by changing the physicochemical properties (i.e., size, shape, and charge) of nanosystems. Furthermore, the so-called “passive targeting” effect can further improve the tumor retention time of nanosystems through surface modification with tumor targeting groups (e.g. peptides, antibodies). Although these approaches prolong the retention time of photosensitizers in tumors, these nanosystems are usually cleared from tumors within one to three days, and it is still insufficient to guarantee effective PDT in many cases. Phototherapy still needs to be performed as soon as possible after the injection of photosensitizers, and large doses or multiple injections of photosensitizers are required for the treatment of large-volume tumors or malignant tumors. However, these will increase the risk of treatment during PDT and lead to significant toxic side effects. Therefore, achieving long-term retention time of photosensitizers at tumor sites is highly desirable, but remains one of the key challenges for PDT.

Improving the stability of NPs is a direct way to improve the tumor retention effect, because NPs are usually easy to aggregate or disintegrate in a complex blood environment, which is one of the main reasons for limitations in tumor retention time.

### SUMMARY

To solve the above technical problems, the present application provides a photosensitizer co-assembly strategy to prolong tumor retention time by greatly improving the stability of nanoparticles (NPs). Retention time of photosensitizer of the present invention at tumor sites has been greatly improved to about 10 days. The photosensitizer synthesizes Bodipy-phenylethynyl anthracene dyad (AN-BDP) by introducing anthracene into BODIPY-meso sites. Due to the strong intermolecular π-π interaction of anthracene, AN-BDP and amphiphilicblock copolymer (distearoyl phosphatidyl ethanolamine-polyethylene glycol (DSPE-PEG₂₀₀₀)) are self-assembled into stable NPs (i.e., AN-BDP NPs). In addition, anthracene can also make AN-BDP excited from a singlet excited state to a triplet excited state and react to O₂ to generate ¹O₂ under the irradiation of light. In case of tail vein injection of AN-BDP NPs, its enrichment and retention time at the tumor sites is up to about 10 days. In a conventional volume-sized mouse tumor model (a tumor initial volume of about 100 mm³), AN-BDP NPs can completely inhibit tumor growth with a single phototherapy injection. In contrast, in a large-volume tumor model (a tumor initial volume of about 350 mm³), only 12% tumor growth inhibition was observed under the same treatment conditions. Since the long tumor retention time, a single injection of AN-BDP NPs can be subjected to 3 times of phototherapy, which significantly improve the treatment effect; while the clinically used photosensitizer Ce6 or Porphyrin NPs encapsulated with the same amphiphilic block copolymer (DSPE-PEG₂₀₀₀) cannot inhibit the growth of large tumors because they are quickly cleared from the tumor sites and only a single phototherapy can be performed under the same conditions. Therefore, improving tumor retention time with stable NPs can be used to treat large-volume tumors multiple times with a single injection, thereby reducing toxic side effects.

The present invention provides a photosensitizer having a structural formula expressed by the general formula I as follows:

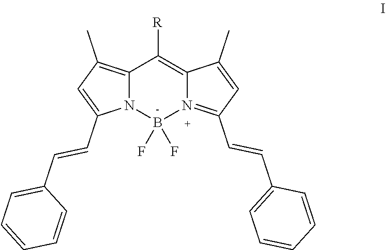

wherein in the formula I, R is selected from:

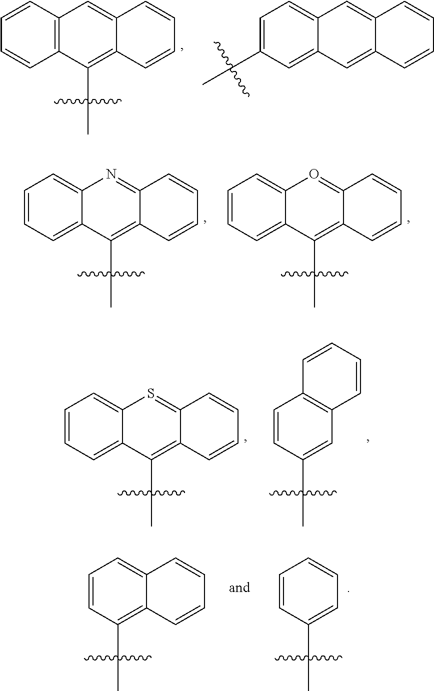

The present invention further provides nanophotosensitizer particles, where the photosensitizer and DSPE-PEG₂₀₀₀ are self-assembled to form nanoparticles.

The present invention further provides a use of the photosensitizer in preparing an antitumor drug, and a use of the nanophotosensitizer particles in preparing an antitumor drug.

Further, in the above technical solutions, the antitumor drug is an antitumor drug for photodynamic therapy.

Further, in the above technical solutions, the antitumor drug is used for multiple photodynamic therapies of a large tumor through a single injection of the photosensitizer or nanophotosensitizer particles.

Further, in the above technical solutions, a retention time of the photosensitizer or the nanophotosensitizer particles at the tumor site is <300 h.

Further, in the above technical solutions, the large tumor is a tumor with a volume of 300-350 mm^{3.}

Further, in the above technical solutions, a frequency of the photodynamic therapy is 1-3 times.

Further, in the above technical solutions, for the large tumor, the treatment effect can be enhanced by one administration and three phototherapies, and the side effects caused by large drug dose and the light dose can be reduced.

### BRIEF DESCRIPTION OF THE DRAWINGS

FIG. 1, panel (a) shows a schematic diagram of the self-assembly of AN-BDP NPs and a intermolecular interaction mode of NPs, and FIG. 1, panel (b) shows AN-BDP NPs used for photodynamic therapy (PDT), which prolonging blood circulation and tumor retention time.

FIG. 2, panel (a) shows an ultraviolet-visible absorption spectrum of AN-BDP and AN-BDP NPs, and FIG. 2, panel (b) shows a fluorescence emission spectrum of AN-BDP and AN-BDP NPs.

FIG. 3, panel (a) shows a lifetime decay curve of AN-BDP in degassed dichloromethane at 630 nm, with a 590 nm pulsed laser as the excitation light source, and FIG. 3, panel (b) shows a molecular structure of 2,6-diiodostyrene BODIPY.

FIG. 4 shows an absorption spectrum attenuation of diphenylisobenzofuran (DPBF) of AN-BDP under 630 nm light irradiation.

FIG. 5 shows an electron paramagnetic resonance (EPR) signal generated by AN-BDP NPs under 630 nm light irradiation, where the red line marks a ¹O₂ singlet oxygen signal indicated by thymopoietin (TMPO).

FIG. 6, panel (a) is a Transmission Electron Microscope (TEM) diagram of AN-BDP NPs, and FIG. 6, panel (b) is a dynamic light scattering (DLS) diagram of AN-BDP NPs.

FIG. 7, panel (a) shows the stability of AN-BDP NPs and Me-BDP NPs at 37° C. and pH 7.4 under the conditions of water, 10 mM PBS, 45 g/LBSA and 90 v % FBS.

FIG. 8 is a snapshot of amorphous AN-BDP NPs (panel (a)) and Me-BDP NPs (panel (b)) obtained by molecular dynamics simulation.

FIG. 9, panel (a) shows imaging of cellular uptake of AN-BDP NPs and Me-BDP NPs in 4T1 cells over incubation time.

FIG. 10 shows the dark toxicity and phototoxicity of AN-BDP NPs on HeLa, HepG-2 and 4T1 cells.

FIG. 11, panel (a) shows a subcellular organelle localization of AN-BDP NPs in 4T1 cells, and FIG. 11, panel (b) shows a detection of ¹O₂ in 4T1 cells.

FIG. 12, panel (a) shows a confocal laser scanning microscopy (CLSM) image of lysosomal disruption in 4T1 cells, where a red light irradiation (630 nm, 27 J/cm²) is performed after cells are incubated with AN-BDP NPs (12 μM), and the proportional scale is 20 μm; FIG. 12, panel (b) shows fluorescence imaging of 4T1 live and dead cells co-stained with calcein AM (green) and propidium iodide (red), where a red light irradiation (630 nm, 27 J/cm²) is performed after cells are incubated with AN-BDP NPs (12 μM), and the proportional scale is 500 μm; FIG. 12, panel (c) shows an analysis of 4T1 cell death pathways using flow cytometry, where a red light irradiation (630 nm, 27 J/cm²) is performed after cells are incubated with AN-BDP NPs (12 μM).

FIG. 13, panel (a) shows fluorescence imaging of 4T1 tumor-bearing mice after tail vein injection of AN-BDP and Me-BDP NPs, and FIG. 13, panel (b) is a time-normalized curve of relative fluorescence intensity of AN-BDP and Me-BDP NPs at mouse tumor sites.

FIG. 14 shows an in-vitro imaging of major organs 3 days later after tail vein injection of AN-BDP NPs, including heart, liver, spleen, lung, kidney and tumor.

FIG. 15, panel (a) shows changes in relative tumor volumes of mice in each group during a treatment of conventional size tumor (200 mW/cm² 630 nm laser radiation for 15 min), FIG. 15, panel (b) shows changes in mean body weights of mice during different treatment periods, FIG. 15, panel (c) is a survival curve of tumor-bearing mice of different groups over a 60-day period, and FIG. 15, panel (d) shows H&E and TUNEL assay analysis of tumor tissues in different treatment groups of mice. The proportional scale: H&E 20 μm, and TUNEL 50 μm.

FIG. 16, panel (a) shows changes in tumor volumes of each group of mice with large tumors (about 350 mm³) during treatment, with tail vein injection (200 ct, 100 cti of Ce6, Porphyrin and AN-BDP NPs, and laser irradiation (635 nm and 200 mW/cm² for 15 min) is performed after tail vein injection, where the Ce6 and Porphyrin groups receive laser irradiation after 8 hours of tail vein injection, the AN-BPD NPs + Light group receives laser irradiation after 24 hours of tail vein injection, the AN-BPD + Light (2) group receives laser irradiation on the 1st and 3rd days of tail vein injection, and the AN-BPD + Light (3) group receives laser irradiation on the 1st, 3rd and 5th days of tail vein injection; difference analysis of statistical data is made based on mean ± SD, and if Student's t-test results show p<0.05, it is considered statistically significant in change (*p<0.05, **p<0.01, ***p<0.001); FIG. 16, panel (b) shows changes in mean body weights of large-volume tumor-bearing mice during different treatment periods; FIG. 16, panel (c) shows tumor images of different groups of tumor-bearing mice; and FIG. 16, panel (d) shows H&E and TUNEL assay analysis of tumor tissues in different treatments. The proportional scale: H&E 100 μm, and TUNEL 50 μm.

FIG. 17 is an in-vivo fluorescence image of mice after tail vein injection of Ce6 and Porphyrin NPs, where dotted circle indicates tumor.

FIG. 18 shows the hemolysis rates of red blood cells (RBCs) treated with different concentrations of AN-BDP NPs.

FIG. 19 shows a histological analysis of H&E staining of normal tissues of mice in each group.

### DETAILED DESCRIPTION OF THE EMBODIMENTS

The following non-limiting embodiments may enable those skilled in the art to understand the present invention more clearly, but not to limit the invention in any way.

Apoptosis detection kits (Annexin V-FITC) and reactive oxygen species assay kits (DCFH-DA) were purchased from Beyotime Biotechnology. Live/dead cell staining kits (Calcein AM/PI) and Hoechst 33342 were purchased from KeyGEN BioTECH. Commercially available subcellular organelle localization dyes such as nuclear dye Hoechst 33324, lysosomal LysoTracker Green DND-26, mitochondrial MitroTracker Green FM, and Propidium Iodide (PI) were purchased from Thermo Fisher Scientific.

Hela (human cervical cancer cells), MCF-7 (human breast cancer cells) and 4T1 (murine breast cancer cells) were purchased from the Institute of Basic Medical Sciences (IBMS).

Example 1 Molecular Design and Synthesis of an AN-BDP NPs Photosensitizer

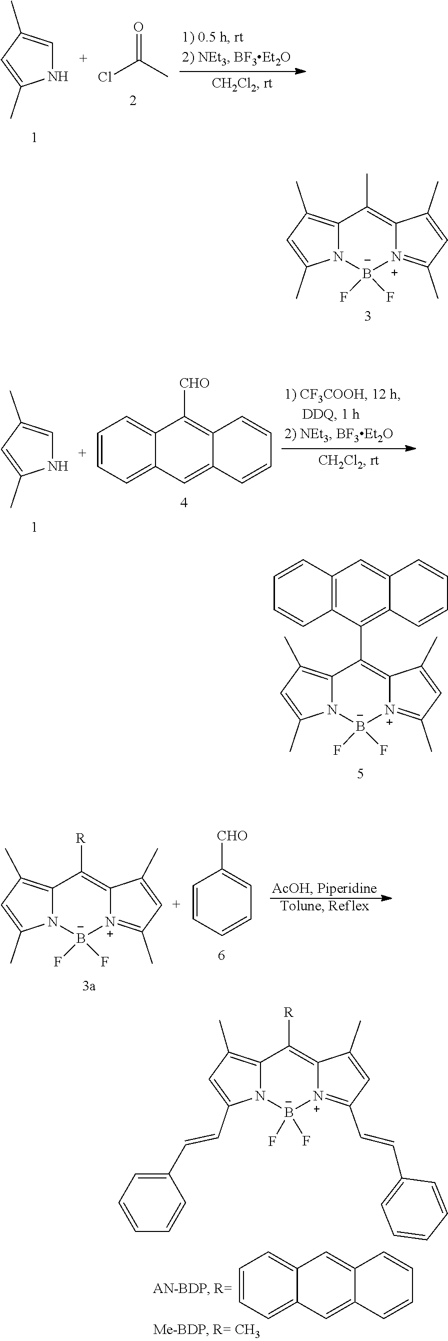

(1) Synthesis of an Intermediate 3

3,5-Dimethyl pyrrole aldehyde (100 mg, 0.81 mM) was dissolved in dry CH₂Cl₂ (15 mL). POCl₃ (124 mg, 0.81 mM) was slowly added to the aforesaid solution under an argon atmosphere at 0° C. The reaction solution was stirred at 0° C. for 1 hour, then NEt₃ (750 mg, 7.4 mm) was added at room temperature and stirred for 4 hours, then BF₃·Et₂O (0.93 ml 7.4 mM) was added to react for 2 hours, and then the solvent was evaporated in vacuum, with EtOAc (200 mL) used for extraction. Then the organic layer after extraction was washed with H₂O (3×50 mL) and dried with anhydrous Na₂SO₄. The crude product was purified by silica gel column chromatography (hexane/EtOAc = 5:1) to obtain the intermediate 3 (red crystal) of 99 mg (49%).

(2) Synthesis of an Intermediate 5

Appropriate amounts of Anthraldehyde (2 mmol) and 2,4-dimethylpyrrole (4 mmol) were dissolved in 250 mL of anhydrous CH₂Cl₂ under N₂ atmosphere. One drop of trifluoroacetic acid (TFA) was added and stirred at room temperature overnight. When results of monitoring by thin layer chromatography (TLC) showed complete consumption of the aldehyde, 2 mmol of 2,3-dichloro-5,6-dicyano-1,4-benzoquinone (DDQ) was added to the CH₂Cl₂ solution and further stirred for 15-20 minutes. The reaction mixture was washed with water, dried with magnesium sulfate, and was filtered and evaporated. The crude compound was roughly purified by alumina peroxide column chromatography to obtain a brown-orange solid. Under nitrogen protection, the brown-orange solid and 4 mL of N,N-diisopropylethylamine (DIEA) were dissolved in 150 mL of anhydrous CH₂Cl₂ (or anhydrous toluene), and the solution was stirred at room temperature for 5 minutes. Then 4 mL of boron trifluoride diethyl etherate (BF₃·BFe₂) was added to the solution and further stirred for 30 minutes. The reaction solution was washed with water and dried with anhydrous MgSO₄, and was filtered and evaporated. The crude compound was purified by silica gel chromatography (CH₂Cl₂/hexane) to obtain an analytically pure sample, which was recrystallized in MeOH to obtain an orange needle.

(3) Synthesis of a Target Compound

Benzaldehyde (0.31 ml, 2.21 mM), 3a (the intermediate 3 or intermediate 5, 0.58 mM), AcOH (0.10 mL) or catalytic amounts of p-Toluenesulfonic acid, and piperidine (0.10 mL) were dissolved in Toluene (10 mL), and a certain amount of activated 4 e molecular sieves was added, with stirring at 80° C. for 5 hours. After quenching with water, the mixture was cooled to room temperature and extracted with CH₂Cl₂. The combined organic layers were washed with sodium chloride solution, and was dried with anhydrous Na₂SO₄ and evaporated. The crude product was purified by TLC (CH₂Cl₂/n-hexane = 3:1) to obtain a final compound:

AN-BDP: ¹H NMR (400 MHz, CDCl₃) NMR (400 MHz, CDClmpound:prJ=8.4, 2 H), 7.96 (d, J=8.6, 2H), 7.85 (d, J=16.3, 2H), 7.66 (d, J=7.5, 4H), 7.55 (d, pound:product was purified by TLC xture TOF-HRMS (EI) (m/z): C₃₁H₄₁BF₂N₂, calculated value: 600.2548; detection value: 600.2543.

¹³C NMR (101 MHz, CDCl₃) δ=152.92, 142.07, 136.63, 136.51, 134.31, 131.34, 130.12, 128.99, 128.82, 128.42, 128.39, 127.61, 127.06, 125.82, 125.27, 119.34, 117.83, 77.33, 77.02, 76.70, 13.60.

Me-BDP: ¹H NMR (400 MHz, CDCl₃) δ=7.72 (m, 2H), 7.66 .63, 136.51, 134.31, 131.34, 130.12, 128.99, 1J=7.1, 2H), 6.73 (s, 2H), 2.50 (s, 6H), 2.30 (s, 3H). TOF-HRMS (EI) (m/z): [M]⁺ calcd for C₂₈H₂₅BF₂N₂: 438.2079; found: 438.2091.

Example 2 Characterization of AN-BDP NPs Photosensitizer Molecules
(1) Ultraviolet-visible and fluorescence spectroscopy characterizations: a dimethyl sulfoxide (DMSO) mother solution of 3 mmol/L AN-BDP and Me-BDPA was accurately prepared, where the test solvent is dichloromethane, the test concentration is 10 μM, and the test temperature is 25° C. The test range of ultraviolet-visible absorption spectrum is 300-800 nm, and the test range of fluorescence emission spectrum is 600-900 nm.

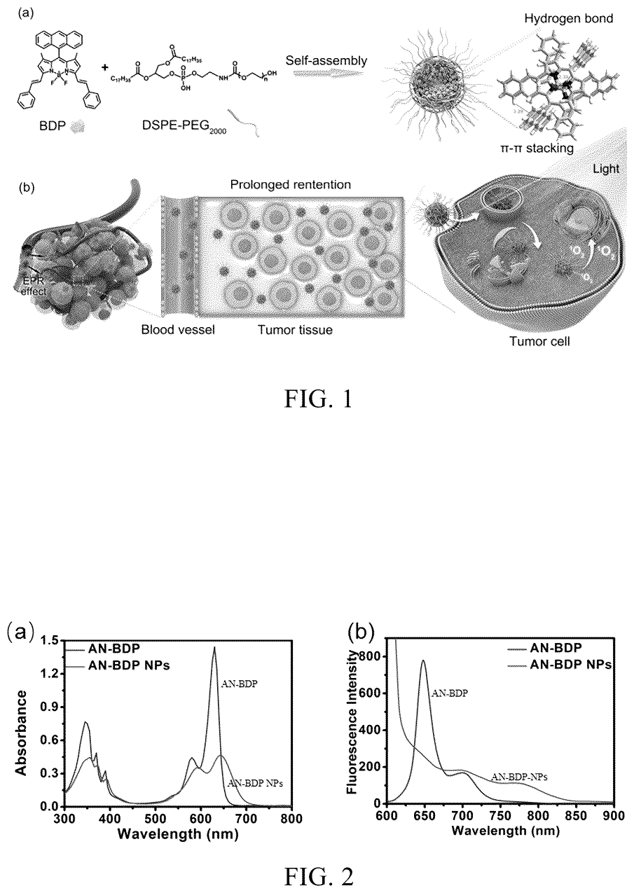

As shown in FIG. 2, the ultraviolet absorption spectrum of 10 μM AN-BDP in dichloromethane shows two adjacent absorption peaks at 578 nm and 630 nm. Whereas, emission of AN-BDP was observed at about 640 nm, indicating that fluorescence imaging is allowed to guide the PDT. However, the absorption spectra of AN-BDP NPs shows two red-shifted peaks at 593 nm and 645 nm, which belong to the typical broad Soret and Q bands, respectively. This indicates that the aggregated AN-BDP is arranged at a certain angle, and the absorption peak of AN-BDP NPs in an aqueous solution shows an obvious red shift compared with that of a monomer in dichloromethane, indicating that AN-BDP is subject to an intermolecular π-π stacking interaction in the assembly process.
(2) Characterization of nanosecond time-resolved transient absorption spectroscopy: a dichloromethane solution with a concentration of 10 μM AN-BDP was prepared, and nitrogen was purged for deoxygenization for 30 min. With a nanosecond time-resolved transient absorption spectrometer, the transient absorption spectra of the two were tested and compared, and the triplet lifetime at 630 nm was fitted according to the data. The excitation light source used in the experiments was a 590 nm pulsed laser.

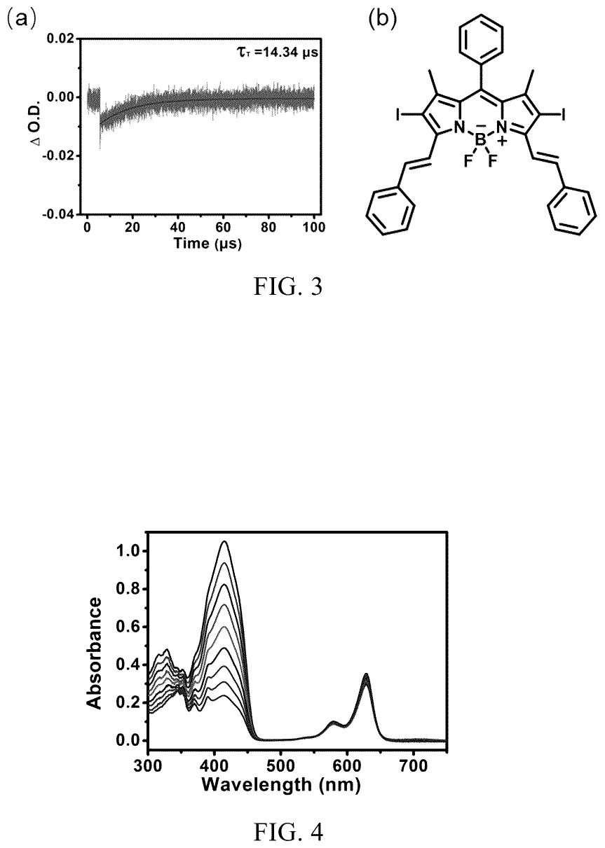

As shown in FIG. 3, panel (a), after excitation of AN-BDP with the 590 nm pulsed laser in deoxygenated dichloromethane solvent, the triplet lifetime of the fitted AN-BDP at 630 nm is 14 μs, which is much longer than that of typical PSs containing heavy atoms (e.g., t=1.7 μs for 2,6-diiodo-distyrene BODIPY). Prolongation of triplet lifetime is more favorable for the energy transfer and electron transfer between a triplet photosensitizer and O₂.
(3) Detection of reactive oxygen species (ROS): to determine whether AN-BDP can generate ROS under red light irradiation, 1,3-diphenylisobenzofuran (DPBF) was used as the ¹O₂ indicator. The absorbance of DPBF at 415 nm was measured, and the capacity to generate ROS was judged according to the change of absorbance at 415 nm.

Operation steps: 2.5 mL of a DPBF CH₂Cl₂ solution with an absorbance of about 1.0 and a dichloromethane solution with an AN-BDP absorbance of about 0.3 were prepared and mixed evenly. A 630 nm LED lamp was used to illuminate a cuvette containing the evenly mixed solution, and a corresponding absorption spectrum was measured every 30 s. The ¹O₂ production level of each sample was evaluated by comparing the changes of absorbance at 415 nm over time. The optical power density of the light source is 5 mW/cm².

As shown in FIG. 4, the absorbance of DPBF at 415 nm is significantly reduced, indicating that AN-BDP has the capacity to generate ROS.
(4) ROS determination: ROS was further verified by electron paramagnetic resonance (EPR) spectroscopy. 5,5-Dimethyl-1-pyrroline-N-oxide (DMPO) was used as a capture agent for O₂-⋅ or ⋅OH, and 2,2,6,6-tetramethyl-1-piperidinyloxy (TMPO) was used as a capture agent for ¹O₂.

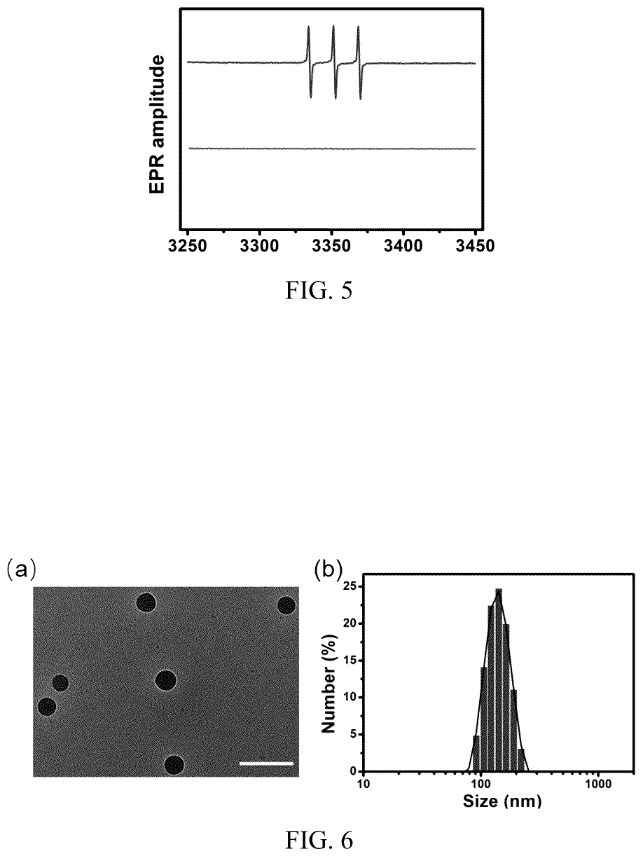

It can be seen from FIG. 5 that the irradiated AN-BDP NPs solution produces a paramagnetic signal matching ¹O₂, indicating that the ROS generated is ¹O₂.

Example 3 Synthesis and Characterization of Nanoparticles (NPs)

(1) Synthesis of NPs

A photosensitizer (AN-BDP or Me-BDP) was dissolved in tetrahydrofuran (THF, 1 mg, 1 mL) to obtain a mixture, then the mixture was added to Milli-Q water containing DSPE-PEG₂₀₀₀ (5 mg, 5 mL) under ultrasonic processing (180 W) and a dispersion liquid was obtained. The obtained dispersion liquid was further kept in ultrasonic processing for 40 minutes to obtain a colloidal dispersion. Thereafter, the colloidal dispersion was dialyzed in the Milli-Q water for 3 days to remove the organic solvent. During the dialysis process, the Milli-Q water was replaced every 6 hours, and finally the dispersion liquid was dialyzed with physiological saline, where the dialysis bag was a regenerated cellulose dialysis bag 3500. The solution in the dialysis bag was collected, that is, the desired NPs.

(2) Characterization of NPs

A certain volume of the NPs solution was added to a sample cell, and the diameter of the NPs was determined by dynamic light scattering (DLS). The morphology of the NPs was measured by transmission electron microscopy (TEM): 2.5 μL of the diluted NPs solution was dropped onto a copper mesh covered with carbon film, and after natural drying, the carbon film was observed with a transmission electron microscope.

AN-BDP was made into NPs by nanoprecipitation. An amphiphilic copolymer (DSPE-PEG₂₀₀₀) was used as the encapsulation matrix. During the assembly process, hydrophobic aromatic planar molecules are randomly accumulated in the core, and hydrophilic PEG chains form a water-soluble shell. The absorption spectra of molecules aggregated in NPs are very different from those in a free state. The size and morphology of the AN-BDP NPs were characterized by using TEM and DLS (as shown in FIG. 6). The TEM image shows that the AN-BDP NPs have spherical morphology with a uniform size of 80-90 nm, while the DLS data indicates that the hydrodynamic diameter of the AN-BDP NPs is 120 nm. The smaller size obtained from TEM measurements may be due to the shrinkage of a hydration layer in a dried TEM sample.

(3) Stability Testing of the NPs

The prepared NPs were diluted to a certain concentration respectively in phosphate buffer solution (PBS), bovine serum albumin (BSA) and fetal bovine serum (FBS) to observe the changes in absorbance of NPs at different times. Changes in absorbance were used to determine the stability of the NPs.

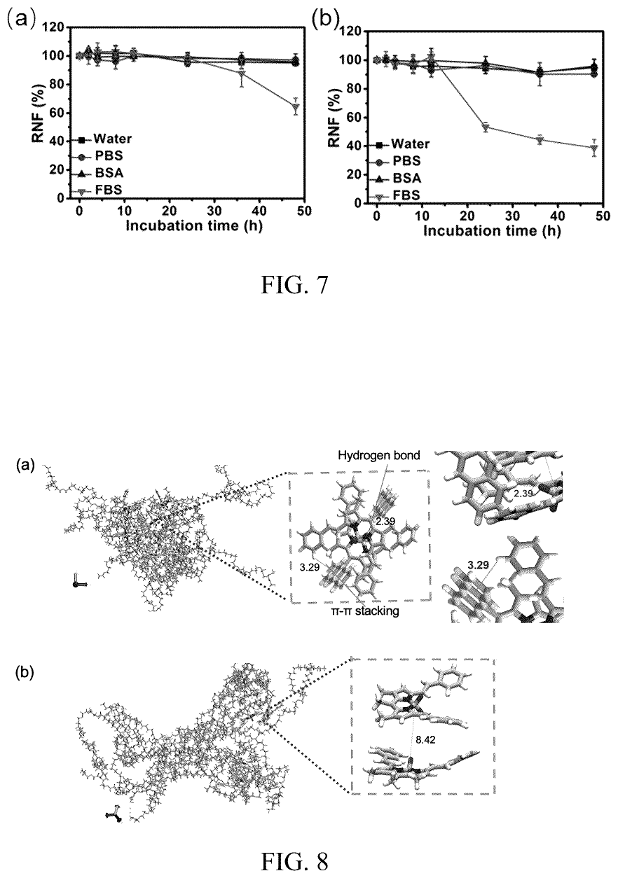

To verify that strong π-π stacking is beneficial to improving the stability of the NPs, BODIPY with meso-methyl (Me-BDP) was synthesized and Me-BDP NPs were prepared for comparison. Then the stability of AN-BDP NPs and Me-BDP NPs under different conditions in vitro was tested. The changes in normalized absorption intensity ratio were used to quantify the nanostructure retention rate (RNF %). After 48 hours of incubation in pure water, PBS buffer and BSA (45 g/L) at 37° C. and pH 7.4, both AN-BDP NPs and Me-BDP NPs had RNF values>95% (FIG. 7), indicating that none of the NPs showed damage. The FBS (90 v/v %) was used to simulate a blood environment. Notably, even a high concentration of FBS cannot significantly induce the damage of AN-BDP NPs. Furthermore, the RNF value of AN-BDP NPs was still about 90% after 36 hours of incubation. In contrast, the RNF value of Me-BDP NPs dropped sharply to 44%. Therefore, AN-BDP NPs show much higher stability than Me-BDP NPs, which is attributed to that the strong π-π stacking interaction between AN-BDP molecules within the hydrophobic block and the protection of PEG chains make AN-BDP NPs have higher serum stability.

Example 4 Dynamics Simulation of AN-BDP NPs and Me-BDP NPs

To reveal the possible mechanism of NPs stability, molecular dynamics (MD) simulations of AN-BDP NPs and Me-BDP NPs were performed by using the GROMACS program (FIG. 8). In fact, π-π stacking is a special spatial arrangement of aromatic compounds, which can be divided into two categories: offset face-to-face (F-type stacking) and edge-to-face (T-type stacking). In an F-type stacking, two aromatic systems form parallel molecular planes with an interplanar spacing of about 3.3-3.8 Å. In a T-type stacking, two aromatic systems are perpendicular to each other. T-type stacking is a CH-π interaction, and existing studies have shown that the T-type stacking is more stable than the F-type stacking. According to snapshots of amorphous aggregates obtained through molecular dynamics (MD) simulations (FIG. 8), the stacking chart of AN-BDP NPs shows that the two adjacent anthracene and BODIPY planes are almost perpendicular to each other, thereby forming intermolecular CH-π interactions (a H-C distance is 3.29 Å, as shown in FIG. 8a). Furthermore, a fluorine bond between the F atom and the anthracene (2.39 2) was found in AN-BDP NPs, which further enhances the stability of the NPs. However, the face-center distance between two Me-BDP molecules is 8.42 Å (FIG. 8b), which exceeds the effective distance of π-π stacking. These results indicate that the interaction between AN-BDP NPs is dominated by CH-π interaction. Therefore, MD calculation results support our conjecture that π-π stacking and hydrogen bonds enhance the stability of the NPs.

Example 5

The murine breast cancer cells 4T1, Hepg-2 cells (human hepatoma cells) and MCF-7 cells (human breast cancer cells) used in the present invention were cultured in a Dulbecco's Modified Eagle Medium (DMEM) containing a 1% mixed solution of penicillin and streptomycin and 10% fetal bovine serum (FBS) at 37° C. in the environment of 2% O₂ and 5% CO₂ until the logarithmic phase, and a cell suspension was prepared after trypsinization for subsequent experiments.

(1) Real-Time Fluorescence Imaging of Cellular Uptake of AN-BDP NPs and Me-BDP NPs

100 μL of a 4T1 cell suspension (1×10⁵ cells/mL) was taken and placed in a confocal dish containing 2 mL DMEM medium, and was cultured at 37° C. in an environment of 5% CO₂ for 24 h until the logarithmic phase. The cultured cells were washed with a phosphate buffer solution (PBS) 3 times before imaging. 2.0 μM of NPs were incubated with 4T1 cells for different times, and then cell nuclei were stained with Hoechst 33342. Fluorescence images were taken by confocal laser scanning microscopy. The excitation wavelength of NPs is 590 nm, and the emission wavelength is detected in a band range of 600-700 nm. The excitation wavelength of Hoechst 33342 is 405 nm, and the detection band range is 425-475 nm.

The uptake and retention time of a photosensitizer in cells is one of the key factors for the antitumor effect of photodynamics. Only when the photosensitizer is effectively enriched and retained in cells, can the reactive oxygen species (ROS) generated after illumination destroy cancer cells and induce cell apoptosis or death, so as to achieve the purpose of inhibiting tumor growth. Cellular uptake of AN-BDP NPs was investigated by using 4T1 tumor cells (FIG. 9). First, 4T1 cells were incubated with Hoechst 33342 for 15 min in the dark. Then, 4T1 cells were incubated with AN-BDP NPs in the dark for different times and imaged by confocal microscopy. Confocal laser scanning microscopy (CLSM) shows that AN-BDPNPs could be rapidly absorbed by 4T1 cells, and the fluorescence intensity increased over the incubation time. Notably, there was still strong red fluorescence after 28 hours of incubation, while the fluorescence of Hoechst 33342 was barely visible. These results indicate that AN-BDP NPs are stable and can remain in cells for a long time. However, the red fluorescence of Me-BDP NPs decreased significantly after 28 h of incubation due to cellular exocytosis. Therefore, strong π-π stacking can make AN-BDP NPs have higher stability and longer retention time, which are both crucial for killing tumor cells during PDT.

(2) Cell Viability Test of AN-BDPNPs

Methyl thiazolyl tetrazolium (MTT) method was used to test the cell viability.

Operation steps: MCF-7, HepG-2 and 4T1 cells were inoculated in 96-well plates (1×10⁴ cells per well and incubated in 100 μL of DMEM). Adherent cells were grown to a density of 80% and incubated with different concentrations of AN-BDP NPs for 4 hours. Then a 630 nm OLED lamp (30 mW/cm²) was used for illuminating for 15 minutes. Meanwhile, under the same experimental conditions, dark cytotoxicity of AN-BDP NPs cultured cells without laser irradiation was further studied. After further incubation for 24 hours, a MTT solution (100 wh of 0.5 mg/mL DMEM) was added to each well and the cells were further incubated for 4 hours at 37° C. Then the medium was carefully removed and 100 μL of dimethyl sulfoxide (DMSO) was added to dissolve the formazan crystals. A Bio-Rad microplate reader was used to measure the absorbance at 490 nm and calculate the cell viability:

$$Cell viability = \frac{{OD}_{Test group} - {OD}_{Blank group}}{{OD}_{Control group} - {OD}_{Blank group}} \times 100 \%$$

The test group is the cell culture group treated with different concentrations of AN-BDP NPs; the blank group is the group only with a medium added; the control group is the cell culture group without AN-BDP NPs; OD is the light absorption of a DMSO solution of formazan at 490 nm.

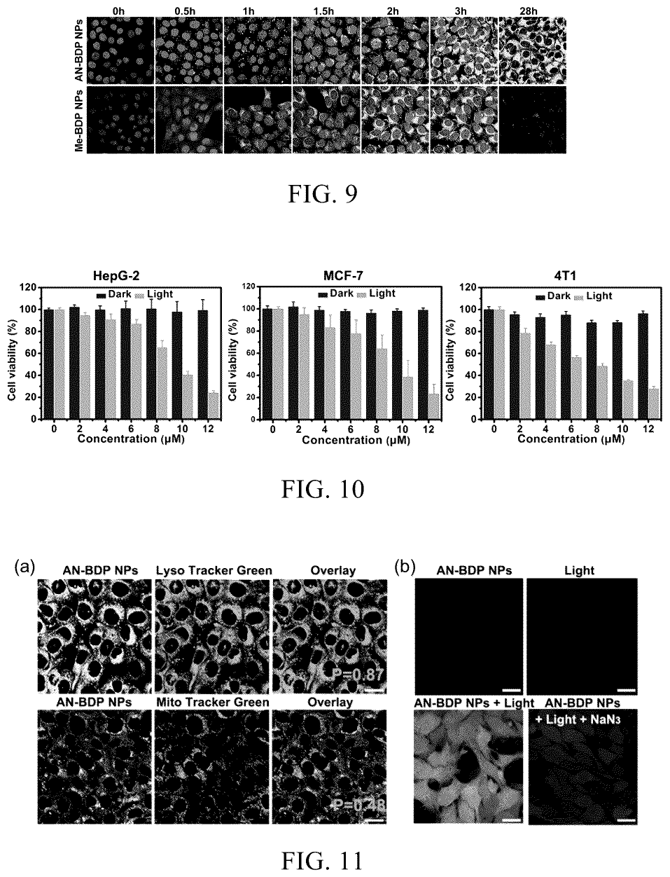

When 12 μM AN-BDP NPs were used under dark conditions, scarcely any cytotoxicity was observed, and the cell viability was above 90%, indicating that AN-BDP NPs had good biocompatibility in the absence of radiation. In contrast, when cells were incubated with the same concentration of AN-BDP NPs under 27 J/cm² light irradiation (630 nm), tumor cell growth was inhibited by about 70%. The cytotoxicity dependent on the concentration and laser power density was observed (FIG. 10). These results indicate that AN-BDP NPs showed a high cytotoxicity under irradiation.

(3) Imaging of Intracellular Singlet Oxygen Production

Operation steps: 4T1 cells were incubated with 5 μM AN-BDP NPs for 3 hours, then incubated with 5 μM of dichlorodihydrofluorescein diacetate (DCFH-DA) for 0.5 hour, the medium was discarded, the incubated product was washed with PBS for three times and 2 mL of serum-free DMEM was added. In the ¹O₂ quenched group, 100 μM of NaN₃ was added to the cells and incubated for 1 hour before the cells were stained with DCFH-DA. Then, the cells were irradiated with 630 nm light (30 mW/cm²) for 15 minutes (27 J/cm²). Confocal fluorescence imaging was used to observe intracellular ¹O₂ levels.

As shown in FIG. 11, panel (b), compared with the control group, under 630 nm LED light illumination, 4T1 cells treated with AN-BDP NPs displayed bright green fluorescence, indicating efficient ROS generation. In contrast, in the groups without light or NPs, no green fluorescence was detected. In addition, the ROS induced by AN-BDP NPs was completely scavenged by NaN₃ (¹O₂ scavenger), indicating that the ROS generated in the cells was ¹O₂. These results clearly indicate that cancer cell death is caused by generation of ¹O₂ in the PDT mediated by AN-BDP NPs.

(4) Subcellular Organelle Colocalization Imaging of AN-BDP NPs

After the 4T1 cells in the confocal dish were grown until the logarithmic phase, 2 μM AN-BDP NPs were added to incubate for 2 h, and then the cells were co-incubated with different commercial dyes for 15 min.

The red fluorescence of AN-BDP NPs was completely superimposed with the green fluorescence of the lysosomal dye LysoTracker Green DND 26 (a commercial lysosomal dye), with a Pearson correlation coefficient of 0.87 (FIG. 11, panel (a)). Therefore, AN-BDP NPs were endocytosed and localized specifically in lysosomes. In contrast, colocalization correlation coefficients of AN-BDP NPs, a commercial mitochondrial dye (MitoTraker Green) and a nuclear dye (Hoechst 33342, FIG. 11, panel (a)) were very low.

(5) A Lysosome Destruction Experiment of AN-BDP NPs Under Red Light Irradiation

Acridine orange, also known as 3,6-bis (dimethylamino) acridine zinc chloride hydrochloride (AO), was used as a lysosomal integrity indicator to visually indicate the destruction of lysosomes by the ¹O₂ generated by excitation of AN-BDP NPs by 630 nm OLED red light.

4T1 cells were inoculated on a 35 mm confocal dish and cultured at 37° C. for 24 hours in a 5% CO₂ environment. Then 4T1 cells were exposed to the following different treatments: the control group, untreated; the light group, irradiated with 630 nm red light at a power density of 30 mW/cm² (27 J/cm²) for 15 minutes; the AN-BDP NPs group, incubated with 10 μM of AN-BDP NPs at 37° C. for 3 hours; the AN-BDP NPs+light group, incubated with 10 μM AN-BDP NPs at 37° C. for 3 hours and irradiated with 630 nm red light at a power density of 30 mW/cm² (27 J/cm²) for 15 minutes. Before the imaging experiment, all cells were incubated with AO (5 Be) for 0.5 h. Finally, observation by a confocal microscope was performed, and fluorescence images were collected for analysis.

A large amount of red fluorescence (white arrow) was observed in the control group, AN-BDP NPs group and light group, indicating that the lysosomes were intact. However, the red fluorescence of AO of AN-BDP NPs disappeared under 630 nm light irradiation (FIG. 12(a)), the cell nucleus was significantly contracted and the cells collapsed (yellow arrow), indicating that the integrity of the lysosome was severely damaged.

(6) An Experiment of Live/Dead Cell Staining Induced by AN-BDP NPs

The 4T1 cells were inoculated in a 35 mm confocal dish and cultured for 24 hours, and after the cells grew until the logarithmic phase, cells in the control group were incubated without any treatment, cells in the light group were irradiated with a 630 nm (30 mW/cm²) LED lamp for 15 minutes, cells in the AN-BDPNPs group were incubated with 12 μM AN-BDP NPs at 37° C. for 4 hours, and cells in the AN-BDP NPs + Light group were incubated with 12 μM AN-BDP NPs at 37° C. for 4 hours, and then were irradiated with a 630 nm (30 mW/cm²) OLED lamp for 15 minutes. The cells were stained with a CalceinAM/PI double staining kit according to the manufacturer's instructions and imaged by a confocal laser scanning microscope (Olympus FV-3000). Calcein: the excitation wavelength is 488 nm, and the emission wavelength for collection is 500-540 nm. PI: the excitation wavelength is 488 nm, and the emission wavelength for collection is 650-690 nm.

Live/dead cell staining experiments were then conducted to confirm the phototherapy effect of AN-BDP NPs under the conditions of 630 nm and 27 J/cm² light irradiation (FIG. 12, panel (b)). Live cells were detected by use of Calcein AM (green), and dead cells were stained by use of propidium iodide dye (red). AN-BDP NPs completely damaged 4T1 cells under the condition of 630 nm and 27 J/cm² light irradiation, as shown by red fluorescence. However, only green fluorescence was observed in the groups with single light and single AN-BDP NPs, indicating that AN-BDPNPs caused strong cytotoxicity under the condition of 630 nm and 27 J/cm² light irradiation.

(7) A Validation Experiment of Cell Death Pathway Induced by AN-BDP NPs

The pathway of cell death induced by ¹O₂ generated by AN-BDP NPs under light irradiation was also investigated. After the 4T1 cells were cultured until the logarithmic phase, 12 μM AN-BDP was added to the cultured cells to incubate for 4 h, and was illuminated with a light source of 630 nm LED (30 mW/cm²) for 15 min. The cells were stained using an Annexin V-FITC/PI Apoptosis Kit and tested by flow cytometry.

Operation steps: 4T1 cells were inoculated in a 35 mm 6-well plate and incubated for 24 hours, and then divided into the following groups: control group with the cells incubated without any treatment, light group with the cells irradiated with a 630 nm (30 mW/cm²) LED lamp for 15 minutes, AN-BDP NPs group with the cells incubated with 12 μM AN-BDP NPs at 37° C. for 4 hours, and AN-BDP NPs+Light group with the cells digested with EDTA-free trypsin after incubation with 12 μM AN-BDP NPs at 37° C. After 4 hours, then centrifuged at 1000 rpm for 5 min to discard the medium. The working solution was prepared and the cells were stained according to the instructions. Finally, states of the cells were tested by flow cytometry.

The control group was similar to the light group and the AN-BDP NPs group, the cell viabilities were above 96%, and no obvious apoptosis signal was detected. In contrast, the percentage of apoptotic cells significantly increased to 35.22% after 630 nm 27 J/cm² light irradiation of AN-BDP NPs (FIG. 12, panel (c)). The results are consistent with the cellular phototoxicity results, indicating that PDT of AN-BDP NPs can effectively induce early apoptosis of tumor cells and eventually lead to cell death.

Example 6

(1) Xenograft Tumor Modeling (4T1 Breast Cancer)

The mice used in this example were all 6-week-old BALB/c female mice purchased from Liaoning Changsheng Biotechnology Co., Ltd. The relevant experimental operations were carried out in accordance with the Guidelines for the Care and Use of Laboratory Animals and the regulations of the National Research Committee, and were approved by the Animal Care and Use Committee of Dalian University of Technology. Document number/ethical approval number is 2018-043. A conventional volume tumor model (an initial volume of about 100 mm³) was established. In addition, in order to verify that long retention time is beneficial to multiple treatments of large tumors or malignant tumors, the present invention also established a large volume (an initial volume of about 350 mm³) tumor model.

(2) In Vivo Targeted Biodistribution and Fluorescence Imaging

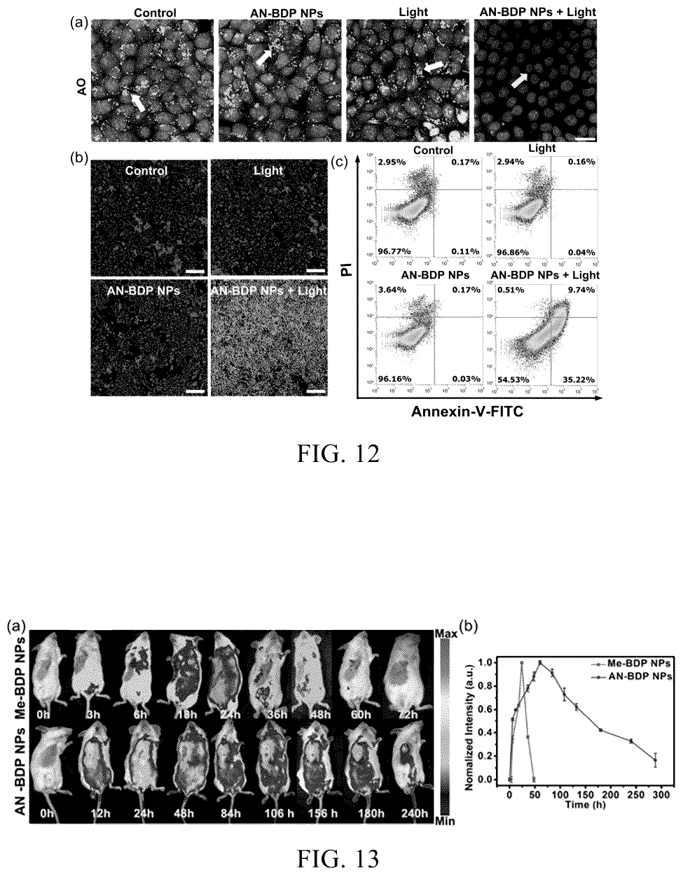

In order to verify that π-π stacking can improve the stability of NPs so as to prolong the tumor retention time of NPs as well as the tumor targeting and optimal treatment time of NPs, the above tumor-bearing mice were selected, AN-BDP NPs or Me-BDP NPs (100 μL, 200 μM) were tail vein injected into the tumor-bearing mice, and fluorescence imaging was observed at different times after injection (FIG. 13, panel (a)). The excitation wavelength is 590 nm, and the emission wavelength is 600-700 nm.

Normalized fluorescence intensity images of time-dependent tumor regions were plotted according to mouse imaging analysis results (FIG. 13, panel (b)). It can be seen from FIG. 13, panel (a) that red fluorescence was first found at the tumor site 18 hours later after injection of Me-BDP NPs, but disappeared 36 hours later, indicating that retention time was limited at the tumor site. In contrast, AN-BDP NPs exhibited a prolonged tumor retention time up to 240 hours, and high-intensity fluorescence lasted from 12 hours to 180 hours, which was much longer than that of previously reported nanophotosensitizers. The excellent tumor enrichment and prolonged retention time of AN-BDP NPs are attributed that the encapsulated AN-BDP has a strong π-π stacking and a protection of the PEG shell, which reduce nonspecific protein adsorption. 240 hours later after injection, almost no fluorescence was detected in other parts of the mice, but a large amount of AN-BDP NPs remained at the tumor sites, showing strong fluorescence. In the process of fluorescence imaging of the mice, the fluorescence signal contrast between the tumor sites and surrounding normal tissues of AN-BDP-injected mice was obvious, the tumor boundary was very clear, and the signal-to-background ratio (SBR) was very high, which can precisely guide PDT. Long tumor retention time allows large tumors or malignant tumors to be treated multiple times with a single injection.

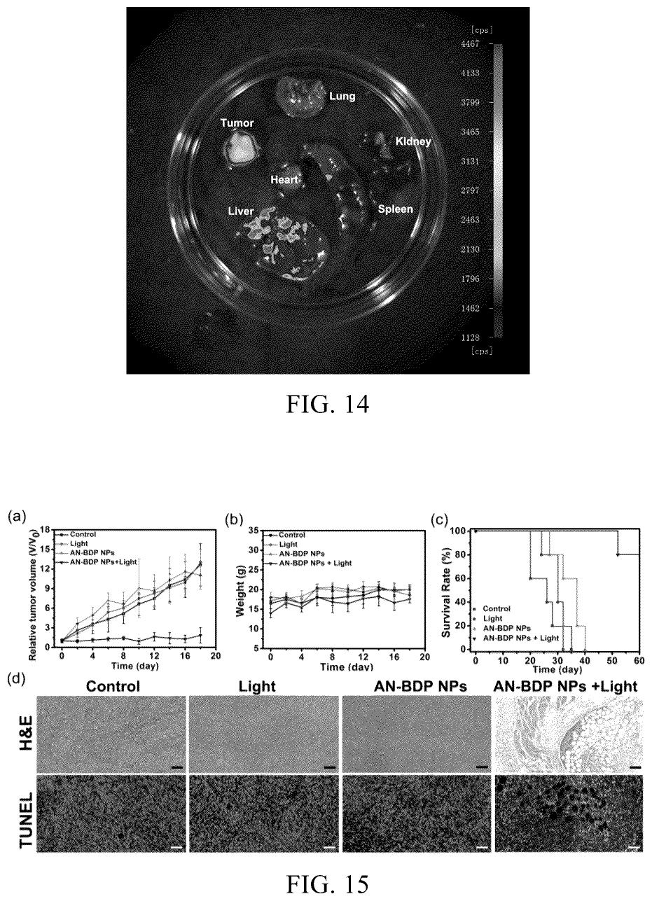

To quantitatively study the biodistribution of major organs, the mice were killed 3 days later after injection of AN-BDP NPs, and tumors and major organs (heart, liver, spleen, lung, and kidney) were collected for imaging. As shown in FIG. 14, AN-BDP NPs were mainly accumulated in tumor tissues, with lower enrichment in liver, which was consistent with the above results of fluorescence imaging of the mice. The high distribution and long-term retention of AN-BDP NPs at tumor sites are beneficial to enhancing PDT.

(3) In Vivo Tumor PDT

The fluorescence imaging-guided in vivo solid tumor targeted PDT experiment was conducted mainly to test the tumor suppression effects of the conventional tumor volume model and the large tumor model respectively.

(i) A Conventional Tumor Volume Inhibition Experiment

In view of excellent tumor enrichment and long retention time of AN-BDP NPs, antitumor activity of AN-BDP NPs in mice with conventional size (initially about 100 mm³) tumors was first investigated. The mice were divided into four groups (n=5), including (1) control group with intravenous injection (i.v.) of physiological saline (100 p), (2) light group with irradiation of 200 mW/cm² 635 nm laser for 15 min, (3) AN-BDP NPs group with tail vein injection of AN-BDP NPs (200 vein injec), and (4) AN-BDP NPs+Light group with tail vein injection of 100 μL of 200 μM AN-BDP NPs, and then 48 hours later with 200 mW/cm² irradiation with 635 nm laser for 15 min. The mice in group (2) treated with physiological saline and laser irradiation (635 nm laser, 200 mW/cm², 15 min) were used for negative control. Body weights and volumes of the mice were measured every other day.

(ii) A Large-Volume Tumor Inhibition Experiment

The present invention evaluated the therapeutic performance of a large tumor model for AN-BDP NPs-injected mice with an initial tumor volume of about 350 mm³. The mice were divided into six groups (n=5), including (1) control group with intravenous injection (i.v.) of physiological saline (100 μL), (2) Ce6 NPs+Light group with tail vein injection of 100 μL of 200 μM Ce6 NPs and irradiation of 635 nm 200 mW/cm² laser for 15 min, (3) Porphyrin NPs+light group with tail vein injection of porphyrin (200 hy, , 100 hyrinn) and irradiation of 200 mW/cm² laser for 15 min, (4) AN-BDP NPs+Light group with tail vein injection of 100 μL of 200 μM LAN-BDP NPs and irradiation of 635 nm 200 mW/cm² laser for 15 min, (5) AN-BDP NPs+Light 2 group with tail vein injection of 100 μL of 200 μM AN-BDP NPs, and irradiations of 635 nm 200 mW/cm² laser for 15 min for twice, and (6) AN-BDP NPs+Light 3 group with tail vein injection of 100 μL of 200 μM AN-BDP NPs, and irradiation of 635 nm 200 mW/cm² laser for 15 min for three times. The fluorescence signal at the tumor site increased in a time-dependent manner, and the fluorescence intensity reached a maximum 8 hours later after injection of Ce6 and Porphyrin. Therefore, groups (2) and (3) received light irradiation (635 nm laser, 200 mW/cm², 15 min) 8 hours later after tail vein injection. Group (4) received light irradiation (635 nm laser, 200 mW/cm², 15 min) 24 hours later after tail vein injection of AN-BDPNPs. Group (5) received light irradiation respectively on the 1st and 3rd days after tail vein injection of AN-BDP NPs, and group (6) received light irradiation on the 1st, 3rd and 5th days after tail vein injection of AN-BDP NPs. Body weight and tumor volume were measured every 2 days for 18 days. Major organs (heart, liver, spleen, lung, and kidney) and tumors were collected after treatment, and examination by hematoxylin and eosin (H&E) staining or TUNEL staining and observation by fluorescence microscopy (EVOS XL Core, Life Technologies, USA) were performed for histological analysis.

In the present invention, the antitumor activity of AN-BDP NPs in the mice with tumors of conventional size (initially about 100 mm³) was first tested. FIG. 15, panel (a) shows the changes in tumor volumes of mice after different treatments. AN-BDPNPs without light irradiation had no significant effect on tumor growth. The same results were also observed in the mice exposed to single light irradiation (FIG. 15, panel (a)), indicating the safety of the light dose (635 nm laser, 200 mW/cm², 15 min) used. Notably, tumor growth of the AN-BDP NPs and Light group was completely inhibited, indicating an excellent tumor suppression effect. The results may be attributed to the fact that AN-BDP NPs were efficiently accumulated at tumor sites, and generated cytotoxic ¹O₂ under light irradiation, thereby achieving efficient PDT. H&E staining and terminal deoxynucleotidyl transferase-mediated dUTP nick end labeling (TUNEL) were used to assess the antitumor effect (FIG. 15, panel (d)). H&E staining was used to observe the morphology of tumor tissue cells. For the PBS, Light and AN-BDP NPs groups, a large number of stromal dense cells were observed with intact nuclei and cytoplasm, indicating that the cells were in good condition. In contrast, in the treatment group (AN-BDP NPs+Light), the cell nuclei were shrunk or fragmented, and scarcely any morphologically intact cells were observed. TUNEL staining was used to mark apoptosis. As expected, the percentage of TUNEL-positive apoptosis found in the AN-BDP NPs+Light group was significantly higher than that of any other group. In all groups, the AN-BDP NPs+Light group showed the optimal therapeutic effect in inducing cell apoptosis. The viability of different groups of mice was also monitored (FIG. panel (c)). The mice treated with AN-BDP NPs+Light showed the strongest viability, and remained 80% viable after 60 days of treatment. All mice in the other groups died within 40 days. The above results show that AN-BDP NPs exhibited an enhanced PDT, and the body weights of mice gradually increased (FIG. 15, panel (b)) without significant changes, indicating that systemic toxicity is negligible.

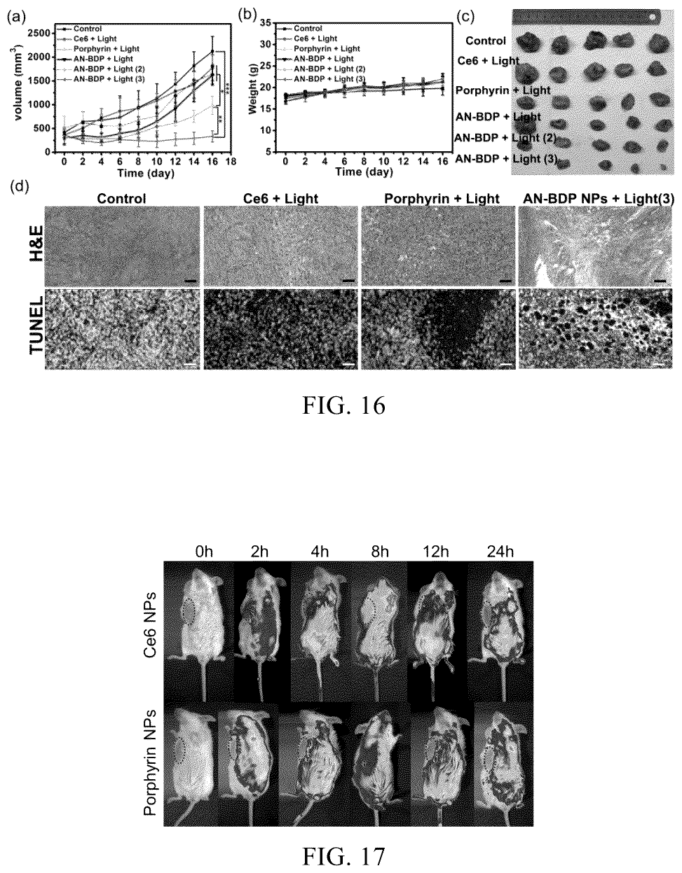

The present invention further evaluated the therapeutic performance of a large tumor model for AN-BDP NPs-injected mice with an initial tumor volume of about 350 mm³ (FIG. 16, panel (a)). In the present invention, two commercial clinical photosensitizers Ce6 and Porphyrin were used for comparison, which were encapsulated in the same amphiphilic copolymer (DSPE-PEG₂₀₀₀) to prepare NPs. 24 hours later after tail vein injection of AN-BDP NPs, 200 mW/cm² 635 nm laser irradiation for minutes could not effectively inhibit tumor growth of mice (AN-BDP NPs group), with only 12% tumor growth inhibition rate, indicating that multiple PDT treatments are necessary for the treatment of large tumors. Since the tumor retention time of AN-BDP NPs is up to about 10 days, multiple PDT treatments can be performed: twice on the 1st and 3rd days, and three times on the 1st, 3rd and 5th days. As expected, after phototherapy with AN-BDP NPs and irradiation twice (AN-BDP NPs+Light 2), tumor growth was inhibited with a tumor inhibition rate increased to 50%. It is worth noting that when the AN-BDP NPs-injected mice in the AN-BDP NPs+Light 3 group were exposed to three times of light irradiation, the tumors thereof were effectively inhibited, and some tumors eventually disappeared (an average tumor inhibition rate was 90%). However, the mice in the Ce6 and porphyrin groups received only one phototherapy under the same conditions, because the NPs exhibited a limited tumor enrichment and short tumor retention time (FIG. 17). Thus, neither Ce6 nor Porphyrin NPs resulted in a potent tumor growth inhibition (12% of tumor inhibition rate), showing the similarity to that of one-time light-treated AN-BDP NPs (FIG. 16, panels (a), (c)). These results clearly demonstrate that due to the enhanced tumor retention effect, AN-BDP NPs can be used for effective tumor PDT on larger tumors by means of single injection and multiple phototherapies. To evaluate the anti-tumor effect for larger tumor-bearing mice, H&E and TUNEL staining were used to observe the morphology of tumor tissues after treatment (FIG. 16, panel (d)). In the control group, a large number of cells with compact interstitium were observed, and the nuclei and cytoplasm were intact, indicating that the cells were in good condition. In addition, it can be observed that only a small number of cells of mice in the Ce6+Light and Porphyrin+Light groups showed the symptoms of cell nucleus shrinkage or fragmentation. In contrast, in the treatment group (AN-BDP NPs+Light 3), the cell nuclei shrank or were fragmented, and scarcely any cells with intact morphology were observed. TUNEL staining was used to mark cell apoptosis. As expected, the TUNEL-positive apoptotic fluorescence signal found in the AN-BDP NPs+Light 3 group was significantly higher than that of any other group. In all groups, the AN-BDP NPs+Light 3 group showed the optimal therapeutic effect in inducing cell apoptosis, indicating that long retention time of photosensitizers in tumors can enhance the PDT effect of the large tumor model.

(4) In Vivo Biosafety Evaluation

First, the biosafety of AN-BDP NPs was assessed based on blood analysis. The specific steps are as follows: 3 mL of fresh sterile defibrated sheep blood was added to 6 mL of a PBS buffer, centrifuging at 1500 rpm was performed for 8 min, and the supernatant was discarded until the supernatant was clear. Then the obtained erythrocytes were used to prepare a 2% erythrocyte suspension. Different concentrations of AN-BDP NPs were co-incubated with an equal volume of 2% erythrocyte suspension for 4 hours at 37° C. and 180 rpm. After the completion of incubation, complete erythrocytes were removed by centrifugation at 1500 rpm for 8 min, and the supernatant was taken to measure the absorbance at 545 nm with a microplate reader.

$$Hemolysis rate = \frac{{OD}_{Test group} - {OD}_{Negative control group}}{{OD}_{Positive control group} - {OD}_{Negative control group}} \times 100 \%$$

The test group is the erythrocyte suspension group treated with different concentrations of AN-BDP NPs; the negative control group is the erythrocyte suspension group incubated with a PBS buffer; the positive control group is the erythrocyte suspension group incubated with deionized water; and OD is the absorbance at 545 nm.

In the present invention, the safety was tested in vivo. Specific steps are as follows: the healthy mice were divided into four groups (n=5), including (1) control group with intravenous injection (i.v.) of physiological saline (100 ph), (2) light group irradiated with 200 mW/cm² 635 nm laser for 15 min, (3) AN-BDP NPs group with tail vein injection of AN-BDP NPs (200 v, 100 v), (4) AN-BDP NPs+Light group with tail vein injection of 100 μL of 200 μM AN-BDP NPs, and then 48 hours later with 200 mW/cm² irradiation with 635 nm laser for 15 min. The whole blood was taken for analysis. The group with physiological saline was used for negative control.

The in vivo safety of AN-BDP NPs was also evaluated by observing the significant changes in body weights of each group of mice during the treatment period, and the tissue staining of major organs after the treatment.

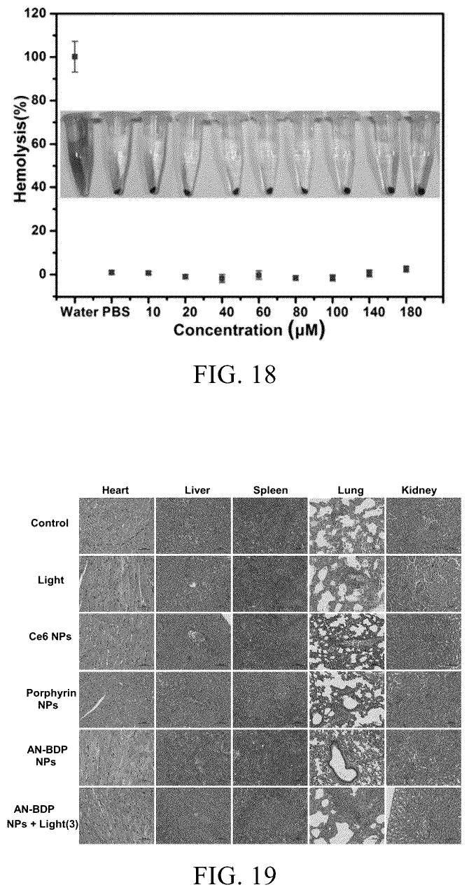

Good biocompatibility is one of the necessary conditions for biomedical materials. First, blood analysis was made to assess the biosafety of AN-BDP NPs. The material with a hemolysis rate of less than 5% is considered to have good biocompatibility. In the present invention, no obvious hemolysis occurred (FIG. 18) even when the concentration of the AN-BDP NPs solution reached up to 200 μM, indicating that the present invention has good biocompatibility to blood.

Second, whole blood testing of the mice was also performed for the present invention, and all measured parameters were within a normal range (Table 1), indicating acute inflammation caused by AN-BDP NPs is negligible and the biocompatibility is good.

TABLE 1
Whole blood cells detection, including RBC, WBC, PLT,
HGB, HCT, MCV, MCH, NEUT, LYMPH, and MONO

| | RBC (M/μL) | WBC (K/μL) | PLT (K/μL) | HGB (g/dL) | HCT (%) |
|---|---|---|---|---|---|
| Reference range | 6.36-9.42 | 0.8-6.8 | 450-1590 | 110-143 | 34.6-44.6 |
| Control | 9.14 | 7.3 | 672 | 144 | 46.7 |
| Light | 9.33 | 7.6 | 380 | 162 | 51.0 |
| AN-BDP NPs | 8.30 | 6.0 | 336 | 133 | 43.1 |
| AN-BDP NPs + Light | 9.34 | 8.3 | 333 | 153 | 49.1 |
| | MCV (fL) | MCH (pg) | NEUT (K/μL) | LYMPH (%) | MONO (%) |
| Reference range | 48.2-58.3 | 15.8-19 | 0.1-1.8 | 55.8-90.6 | 1.8-6.0 |
| Control | 51.1 | 15.7 | 3.2 | 49.1 | 7.5 |
| Light | 54.7 | 17.3 | 2.4 | 62.1 | 6.4 |
| AN-BDP NPs | 52.0 | 16.0 | 1.3 | 75.0 | 3.6 |
| AN-BDP NPs + Light | 52.6 | 16.3 | 1.4 | 79.8 | 3.5 |

Notably, the mice in different groups gained weight slowly, but no additional side effects were observed throughout the experiment (FIG. 15, panel (b) and FIG. 16, panel (b)). Finally, a histopathological assessment of major organ tissues of mouse was made after the end of the treatment cycle. As shown in FIG. 19, H&E staining of all tissues showed no significant pathological changes in all groups, indicating that the AN-BDP NPs have good biocompatibility.

In conclusion, the present invention has demonstrated that strong π-π stacking-stabilized nanophotosensitizers are promising, which can prolong the blood circulation and tumor retention time of nanophotosensitizers during PDT. AN-BDP NPs exhibites excellent stability and may stay at the tumor sites for up to 10 days, which is longer than that of any other existing photosensitizers. In a mouse tumor model for routinely tested tumor volume size (about 100 mm³), single injection and single phototherapy can completely inhibit tumor growth. More importantly, the nanophotosensitizer of the present invention capable of prolonging tumor retention time may be successfully applied to a mouse tumor model of larger tumors (about 100 mm³) for treatment by means of single injection and multiple phototherapies. Our work highlights that rationally designed nanophotosensitizers may be used to achieve multiple PDTs through a single injection, and enhance the PDT effect for the treatment of large-volume tumors or malignant tumors. The present invention provides an available, simple and convenient method to prolong the tumor retention time of the photosensitizer, and further promote the clinical translation of nanomedicine.

1. A photosensitizer having a structural formula of formula I: 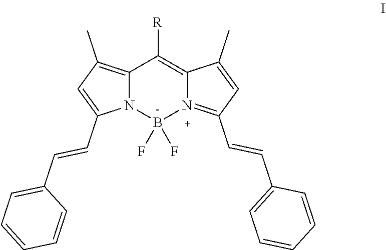
wherein R is selected from:

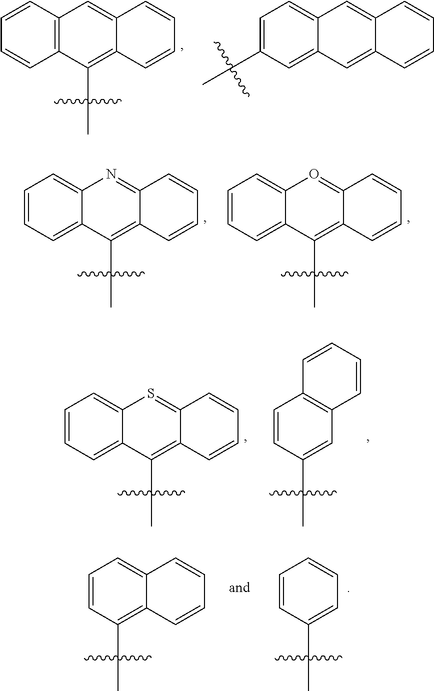

2. A nanophotosensitizer particles formed by the photosensitizer according to claim 1 and DSPE-PEG₂₀₀₀ by self-assembled.

3. An antitumor drug comprising the photosensitizer according to claim 1.

4. An antitumor drug comprising the nanophotosensitizer particles of claim 2.

5. The antitumor drug of claim 3 is an antitumor drug for photodynamic therapy.

6. A method for treating a large tumor, comprising administering a single injection of the photosensitizer of claim 1.

7. The method of claim 6, wherein a retention time of the photosensitizer at the tumor site is <300 h.

8. The method according to claim 6, wherein the large tumor is a tumor with a volume of 300-350 mm³.

9. The method according to claim 6, wherein a dosage for treating the large tumor comprises one administration and three phototherapies.

10. The antitumor drug of claim 4 is an antitumor drug for photodynamic therapy.

11. A method for treating a large tumor, comprising administering a single injection of the nanophotosensitizer particles of claim 2.

12. The method of claim 11, wherein a retention time of the nanophotosensitizer particles at the tumor site is <300 h.

13. The method according to claim 11, wherein the large tumor is a tumor with a volume of 300-350 mm³.

14. The method according to claim 11, wherein a dosage for treating the large tumor comprises one administration and three phototherapies.
